(19) United States
(12) Patent Application Publication
(10) Pub. No.: US 2015/0366657 A1
(43) Pub. Date: Dec. 24, 2015

(54) CORNEAL IMPLANT EDGES AND METHODS OF USE

(71) Applicant: REVISION OPTICS, INC., Lake Forest, CA (US)

(72) Inventor: Guru Dutt SHARMA, Lake Forest, CA (US)

(21) Appl. No.: 14/427,510

(22) PCT Filed: Nov. 21, 2012
(86) PCT No.: PCT/US2012/066198

§ 371 Date: Mar. 11, 2015

(51) Int. Cl. A61F 2/14

(52) U.S. Cl. CPC A61F 2/142; A61F 2230/0071

(57) ABSTRACT

Corneal implants and methods for reducing gap formation between corneal tissue after implanting the corneal implant. In some embodiments the corneal implants include a peripheral portion adapted to be reconfigured to more closely conform to the shape of a corneal bed. In some embodiments the corneal implant is adapted to be reconfigured such that when a corneal flap is positioned over the corneal implant gap formation is reduced or eliminated.

### CROSS REFERENCE TO RELATED APPLICATIONS

This application is related to and incorporates by reference herein the following patent applications: U.S. Pat. No. 6,102,946, issued Aug. 15, 2000; U.S. Pat. No. 6,361,560, issued Mar. 26, 2002; U.S. Pat. No. 7,776,086, issued Aug. 17, 2010; U.S. Pub. No. US 2005/0246016 A1, published on Nov. 3, 2005; U.S. Pub. No. 2011/0218623, published on Sep. 8, 2011; U.S. Pat. No. 8,057,541, issued Nov. 15, 2011; U.S. Pub. No. 2008/0262610, published Oct. 23, 2008; U.S. Pub. No. 2009/0198325, published Aug. 6, 2009.

### INCORPORATION BY REFERENCE

All publications and patent applications mentioned in this specification are herein incorporated by reference to the same extent as if each individual publication or patent application was specifically and individually indicated to be incorporated by reference.

### BACKGROUND

Corneal inlays are positioned within corneal tissue and used to correct one or more types of refractive errors. Some corneal inlays are adapted to treat presbyopia by creating multiple focalities within the cornea (see U.S. Pat. No. 5,336,261). Some corneal inlays have a generally annular configuration and are positioned within corneal tissue to correct presbyopia by creating regions of near and far vision within the pupil (see U.S. Pat. App. Pub. No. 2001/0027314). Some corneal inlays have bodies that are adapted to be positioned within corneal tissue to change the curvature of the anterior surface of the cornea (see U.S. Pat. App. Pub. No. 2001/0027314, U.S. Pat. No. 5,123,912, and U.S. Pat. App. Pub. No. 2011/0218623).

These and other inlays can be positioned within corneal tissue by first creating a corneal flap, either mechanically or with a laser, and lifting the flap to expose a corneal bed onto which the inlay is to be positioned. Once the inlay is positioned in place on the corneal bed, the flap is placed back down over the corneal inlay. The corneal flap tissue in the flap will subsequently heal with the corneal bed tissue.

When the flap is repositioned on some inlays, the configuration of the peripheral portion of the inlays creates a microscopic “gap,” or “space” created between the corneal flap tissue and the corneal bed tissue.

Not only can the gap interfere with normal corneal healing, but it is also a potential space in which cellular byproducts, such as but not limited to lipid deposits, can accumulate. The deposits can initially appear as a circular faint whitish ring around the circumference of the inlay, interfering with perfect transparency, and can develop into more pronounced edge deposits and peripheral deposits. The location of edge deposits may also serve as a nucleation site for additional haze development. In some patients the haze can occur initially or in later post-operative stages. The gap also prevents complete healing up to the edge of the implant.

Edge deposit is generally a benign finding and generally does not progress to actual haze over the inlay under most eye conditions and good corneal health. However, suboptimal corneal conditions causing corneal insults or injury may trigger edge deposits to progress in a small proportion of patients over and/or under the inlay. It may be of concern for long-term stability and clinical efficacy of the corneal inlay being implanted. In some patients trace haze may also induce a drop in uncorrected distance, intermediate, and near vision, as well as disturbing visual symptoms.

As used herein, references to “edge deposit” refer to any of the following: edge build-up, visually observable indications such as a whitish ring around the inlay, and the accumulation of cellular byproducts near the periphery of the inlay. These are, by themselves, benign findings and do not affect the patient's uncorrected or corrected vision.

Peripheral inlay haze of clinical significance is currently treated with steroids. Steroids can, however, induce a rise in intraocular pressure, glaucoma, and optic nerve damage. Steroids can also induce early cataracts. Additionally, even if the haze is reduced with steroids, the haze can reoccur. It is preferred to reduce or eliminate the haze before it occurs.

### SUMMARY OF THE DISCLOSURE

One aspect of the disclosure is a method of reducing gap formation between corneal tissue after the implantation of a corneal implant, comprising creating a corneal bed, positioning a corneal implant on the corneal bed, and reconfiguring a peripheral portion of the corneal implant from a first configuration to a second configuration.

In some embodiments deforming the peripheral portion comprises reconfiguring the peripheral portion so that it more closely conforms to the corneal bed. The reconfiguring step can comprise reconfiguring the peripheral portion so that a first surface in the peripheral portion conforms more closely to the corneal bed when the peripheral portion is in the second configuration.

In some embodiments the method further comprises creating a corneal flap, lifting the corneal flap to create the corneal bed, and placing the flap back down over the corneal implant after positioning the corneal implant on the corneal bed, wherein placing the flap back down over the corneal implant reconfigures the peripheral portion of the corneal implant from the first configuration to the second configuration such that gap formation between the flap, the corneal bed, and the cornea implant is reduced upon the reconfiguration to the second configuration.

In some embodiments reconfiguring a peripheral portion comprises changing the orientation of a first surface from a first orientation to a second orientation so that it more closely conforms to the corneal bed. In the first orientation that first surface can be at an angle relative to a posterior surface of the corneal implant. In the first configuration the surface can be disposed at an angle less than 90 degrees relative to an axis that is orthogonal to an optical axis of the corneal implant. In the first configuration the surface can be disposed at an angle between about 10 degrees and about 80 degrees relative to an axis that is orthogonal to an optical axis of the corneal implant.

In some embodiments reconfiguring the peripheral portion comprises changing the orientation of a posterior beveled surface relative to a posterior surface of the corneal implant so that the beveled surface is more closely aligned with the posterior surface.

In some embodiments in the first configuration a peripheral edge of an anterior surface of the corneal implant is further from the corneal bed than the peripheral edge of the anterior surface in the second configuration.

One aspect of the disclosure is a corneal implant with a peripheral portion adapted to reduce gap formation between corneal tissue after implantation of the corneal implant, the corneal implant comprising a body comprising a posterior surface adapted to be placed on a corneal bed, an anterior surface, and a peripheral portion comprising a first surface adapted to be reconfigured from a first orientation relative to the posterior surface to a second orientation relative to the posterior surface, wherein in the second orientation the first surface is more closely aligned with the posterior surface than in the first orientation.

In some embodiments the first surface extends from the posterior surface and is disposed at an angle relative to the posterior surface in the first orientation. In the second orientation the first surface can be disposed at a second angle relative to the posterior surface, wherein the second angle is less than the first angle. The second angle can be substantially zero.

In some embodiments in the first orientation the first surface is disposed at an angle less than 90 degrees relative to an axis orthogonal to an optical axis of the inlay. In the first orientation the first surface can be disposed at an angle between about 10 degrees and about 80 degrees relative to an axis that is orthogonal to an optical axis of the corneal implant. In the first orientation the first surface can be disposed at an angle between about 15 degrees and about 60 degrees relative to an axis that is orthogonal to an optical axis of the corneal implant. In the first orientation the first surface can be disposed at an angle between less than 45 degrees relative to an axis that is orthogonal to an optical axis of the corneal implant.

In some embodiments the corneal implant is made of a hydrogel material.

In some embodiments the peripheral portion comprises an edge thickness of between about 10 and about 20 microns.

In some embodiments the peripheral portion comprises an edge thickness that is less than about 30 microns.

In some embodiments the peripheral portion is adapted to be reconfigured when the corneal implant is implanted within corneal tissue less than or equal to about 50% of the depth of the corneal stroma.

One aspect of the disclosure is a corneal implant adapted to reduce gap formation between corneal tissue after implanting the corneal implant, comprising a body comprising a posterior surface adapted to be positioned on a corneal bed, a first surface disposed relative to the posterior surface at a first angle less than 90 degrees and greater than 0 degrees relative to an axis that is orthogonal to an optical axis of the body, and an anterior surface, wherein the first surface is adapted to change orientation when implanted within corneal tissue such that it is at a second angle relative to the axis, wherein the second angle is less than the first angle.

One aspect of the disclosure is a corneal implant adapted to reduce gap formation between corneal tissue after implanting the corneal implant, comprising a body with a posterior surface adapted to be positioned on a corneal bed, an anterior surface, and a posterior portion that is configured to be reconfigured from a first configuration to a second configuration upon implantation within corneal tissue, wherein the peripheral portion comprises a first surface that is configured to more closely conform to the corneal bed when the peripheral portion is in the second configuration than when the peripheral portion in the first configuration.

### BRIEF DESCRIPTION OF THE DRAWINGS

FIG. 1 illustrates an example of edge deposits in a gap formed as a result of a corneal implant being positioned within a cornea.

FIG. 2 illustrates an example of edge haze formed as a result of a corneal implant being positioned within a cornea.

FIG. 3A-3B illustrates an exemplary gap formed after the implantation of an exemplary corneal implant.

FIG. 4A-4B illustrates an exemplary gap formed after the implantation of an exemplary corneal implant.

FIG. 5A-5C illustrates an exemplary corneal implant with a peripheral portion adapted to reduce gap formation.

FIGS. 6A-6D illustrate an exemplary method of positioning the implant from FIGS. 5A-5C is a cornea and reconfiguring a peripheral portion of the implant.

### DETAILED DESCRIPTION

The present disclosure generally describes corneal implants, such as corneal inlays, that are configured to reduce the occurrence of edge deposits following their implantation. In some embodiments the corneal implants are corneal inlays that are adapted to be reconfigured from a first configuration to a second configuration to reduce the occurrence of edge deposits following their implantation. In some embodiments the implant has a peripheral region that is adapted to be reconfigured to reduce the occurrence of edge deposits following their implantation. The region of the implant adapted to be reconfigured need not be limited to “peripheral” regions, however. In some embodiments the region of the implant adapted to be reconfigured can be considered an “edge” region of the implant. “Reconfiguring” or a derivative thereof as used herein includes changing a position or orientation of the portion being reconfigured, and includes changing or maintaining the general shape of the portion that is being reconfigured, including changing or maintaining the shape of one or more surfaces thereof.

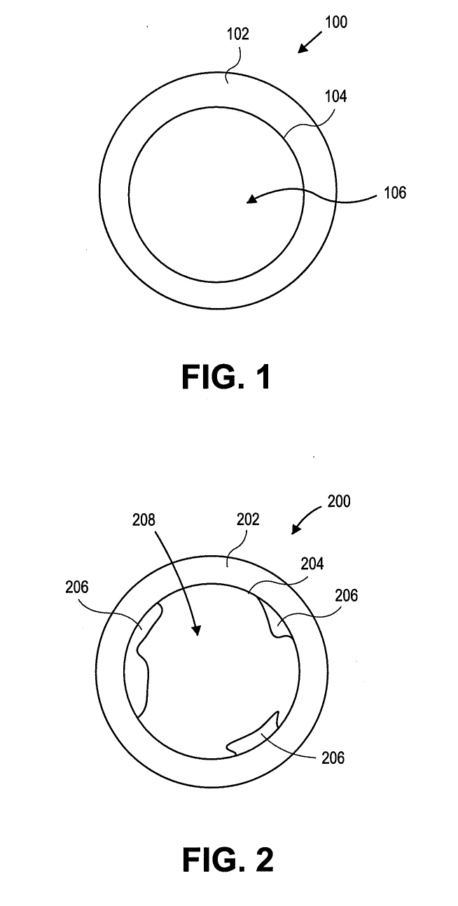

As set forth above, the gap between corneal flap and bed created by some corneal implants not only has the potential to interfere with normal corneal healing, but it is also a potential space in which cellular byproducts, such as but not limited to lipid deposits, can accumulate. The deposits can initially appear as a circular faint whitish ring around the circumference of the inlay and can develop into more pronounced edge deposits and peripheral deposits. FIG. 1 illustrates a portion of eye 100 in which pupil 102 is shown. The corneal implant is positioned within the cornea at location 106, and faint ring 104 created by edge deposits is shown at the periphery of the implant. FIG. 2 illustrates a portion of eye 200 in which pupil 202 is shown. Faint ring 204 is shown, as are regions of peripheral haze 206. The peripheral haze 206 does not affect the vision of the patient.

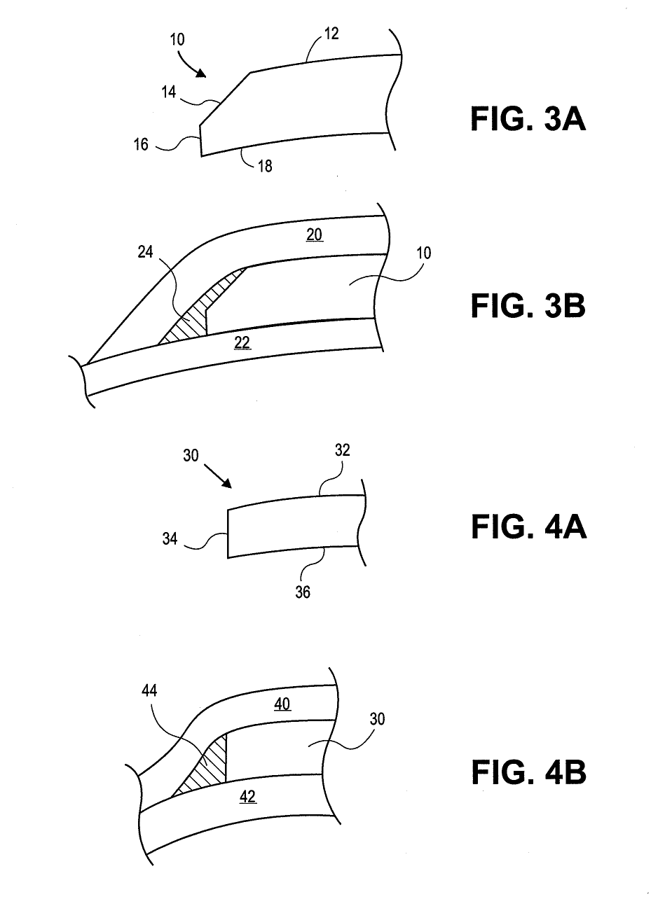

FIG. 3A illustrates a portion of corneal inlay 10 that includes anterior surface 12, beveled anterior surface 14, edge surface 16, and posterior surface 18. FIG. 3B illustrates inlay 10 positioned on corneal bed 22 and under corneal flap 20. FIG. 3B illustrates gap 24 that is created between corneal flap 20, corneal bed 22, and inlay 10 when the flap is placed back down on top of the inlay. The peripheral configuration of the corneal inlay in this embodiment does not allow for complete flap apposition with the bed tissue, which results in the formation of gap 24. The presence of gap 24 prevents corneal tissue interface healing at a location just peripheral to the inlay.

FIG. 4A illustrates an alternative bevelless inlay 30 that includes anterior surface 32, edge surface 34, and posterior surface 36. FIG. 4B illustrates inlay 30 positioned on corneal bed 42, with flap 40 positioned back down on inlay 30. Gap 44 is formed between flap 40, bed 42, and inlay 30 after flap 40 is placed back down on top of inlay 30. The implantation of inlay 30 may result in less of a gap (and therefore less edge deposits) than the embodiment in FIGS. 3A and 3B, but the edge deposits may still be evident in some patients. The inlay in FIGS. 4A and 4B can be used to treat presbyopia, such as is described in more detail in U.S. Pub. No. 2011/0218623, published on Sep. 8, 2011; U.S. Pat. No. 8,057,541, issued Nov. 15, 2011; U.S. Pub. No. 2008/0262610, published Oct. 23, 2008; and U.S. Pub. No. 2009/0198325, published Aug. 6, 2009.

This disclosure generally describes corneal inlays that are adapted to reduce or eliminate the gap between the corneal flap tissue and the corneal bed tissue at the interface with the corneal inlay. By reducing or eliminating the gap, the present disclosure reduces or eliminates the occurrence of edge deposits that may lead to peripheral haze.

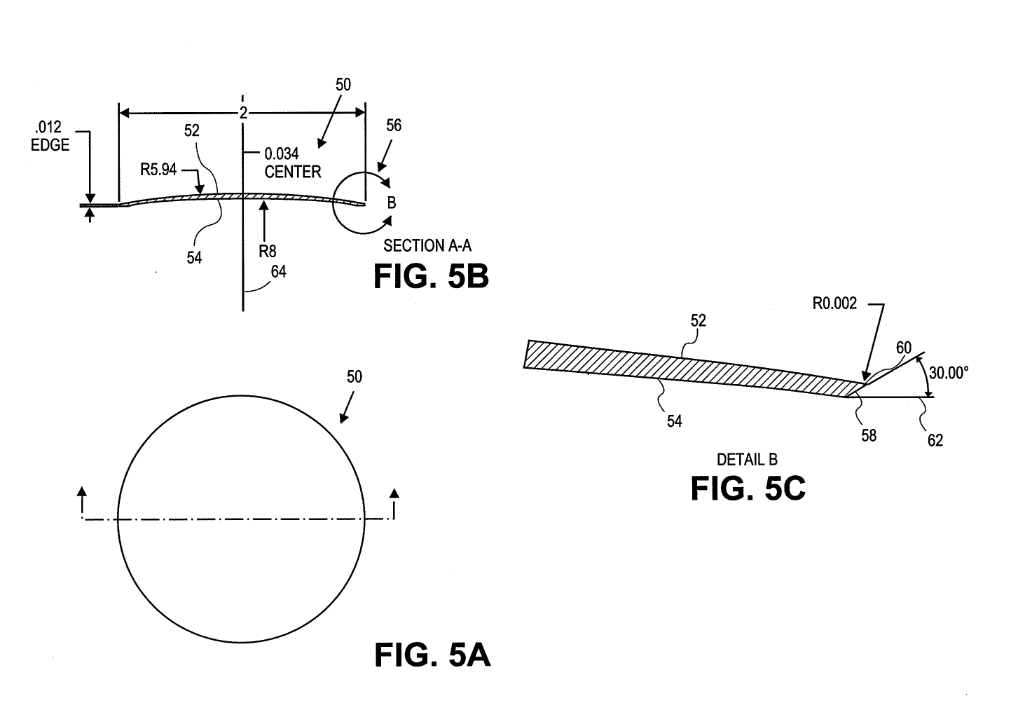

FIGS. 5A-5C illustrate a corneal inlay that is adapted to reduce or eliminate a gap between corneal flap tissue and corneal bed tissue when the inlay is positioned within corneal tissue. FIG. 5A illustrates a top view of inlay 50, while FIG. 5B illustrates section A-A shown in FIG. 5A. FIG. 5C illustrates the peripheral, or edge, portion 56 of the inlay shown in detail “B” from FIG. 5B. As can be seen in FIG. 5B and 5C, inlay 50 includes anterior surface 52, posterior surface 54, and bevel surface 58 extending from posterior surface 54. Bevel surface 58 is shown at a 30 degree angle from axis 62, which is considered orthogonal to the optical axis 64 of the inlay, shown in FIG. 5B. The optical axis passes through the apex of the inlay, and is generally parallel to the light entering the eye that is focused on the retina.

As shown in FIG. 5B, the thickness of the peripheral edge of the inlay is about 12 microns. The thickness of the inlay along the optical axis is about 34 microns. The radius of curvature of anterior surface 52 is about 5.94 mm. The radius of curvature of posterior surface 54 is about 8 mm. These dimensions are merely exemplary, however, and the disclosure is not limited to these dimensions.

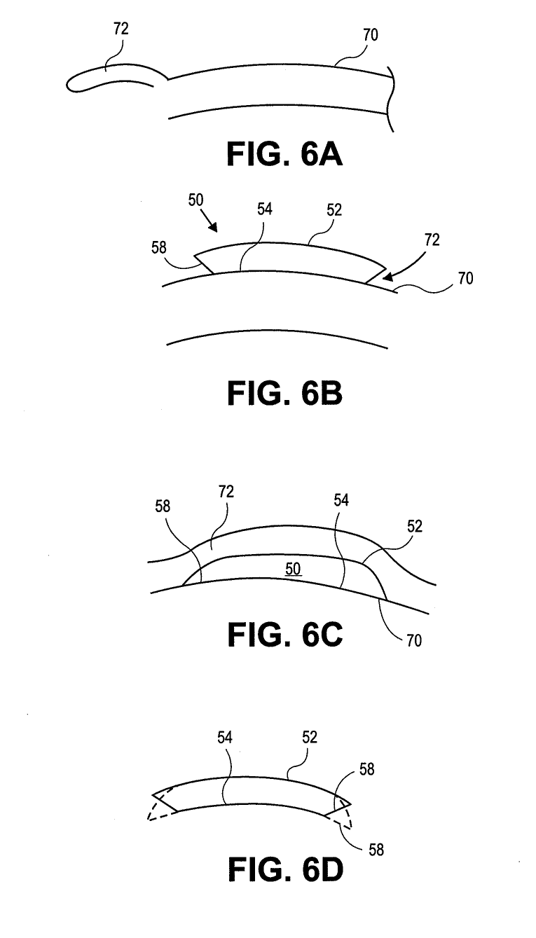

FIGS. 6A-6D illustrate an exemplary implantation procedure of inlay 50 shown in FIGS. 5A-5C. A corneal bed is first prepared by either making a flap and lifting the flap to expose the corneal bed (generally described above), or by creating a pocket within the corneal tissue. There are known techniques for creating a corneal pocket, such as those described in, for example, U.S. Pub. No. US 2012/0046680, published Feb. 23, 2012. FIGS. 6A-6D illustrate a method of use in which a flap is created. FIG. 6A illustrates corneal bed tissue 70 after flap 72 has been created and separated from the corneal bed tissue. Next, inlay 50 is positioned on corneal bed 70, as is shown in the close up view of FIG. 6B (flap not shown). Gap 72 exists between the bevel surface 58 and corneal bed tissue 70 when the inlay is positioned on the corneal bed. Next, flap 72 is laid back down on top of the inlay, as shown in FIG. 6C. As flap 72 is laid back down, the peripheral portion of the inlay is reconfigured from a first configuration, as shown in FIG. 6B, to a second configuration, as shown in FIG. 6C. Bevel surface 58 is reconfigured such that it conforms to corneal bed tissue 70. The periphery of anterior surface 52 is also slightly reconfigured such that it extends more towards the corneal bed than the configuration shown in FIGS. 6C. FIG. 6D illustrates the initial configuration of the peripheral portion of the inlay in solid lines, while the second configuration is shown in broken lines. The corneal tissue is not shown for clarity.

Creating apposition between the periphery of the inlay and corneal bed tissue reduces or eliminates the gap and therefore the occurrence of edge deposits. As it relates to FIGS. 5A-5C, the angle between beveled surface 58 and axis 62 is greater in the first configuration than in the second configuration. In the method shown in FIGS. 6A-6D, the angle between beveled surface 58 and axis 62 approximates zero, but need not be exactly zero, in the second configuration. Additionally, in the second configuration surface 58 may be in general alignment with posterior surface 54, as is shown in FIG. 6D.

It would be beneficial to be able to manufacture a corneal implant such that the manufactured configuration is the second configuration shown with the broken lines. This would avoid having to reconfigure the periphery of the inlay after implantation. It would be ideal to manufacture the inlay such that the anterior surface meets the posterior surface in as sharp an edge as possible. It can be, however, very challenging to manufacture in this way.

To overcome this challenge, the inlay in the embodiment in FIGS. 5A-5C and shown in use in FIGS. 6A-6D is adapted to be reconfigured after initial positioning such that the peripheral portion assumes the desired peripheral configuration. The inlay includes a peripheral region that is adapted to be reconfigured, without damage, to produce a desired peripheral configuration that will eliminate or reduce the gap that results from alternative peripheral designs. In this manner the edge deposits as well as the possibility of trace haze are reduced or eliminated and it enhances the biocompatibility and long-term stability of the implant.

The current disclosure describes a corneal inlay that is adapted to be reconfigured after initially positioning on a corneal bed such that a post positioning configuration reduces or eliminates edge deposit formation around the periphery of the inlay. The current disclosure also describes methods of positioning an inlay within corneal tissue and reconfiguring the peripheral region such that the peripheral region better conforms to the corneal bed. As shown above, the bevel surface is reconfigured towards the surface of the corneal bed. The beveled surface is reconfigured such that it is closer to parallel with the corneal bed surface than it was before it was reconfigured.

In the exemplary embodiment in FIGS. 5A-5C, the edge of the inlay is about 12 microns thick. If the edge is too thick or thin it may not be able to be reconfigured by the force of the corneal tissue. The reconfiguration may also depend on how deep the inlay is positioned and the inlay material. An edge thickness of about 12 microns allows the peripheral portion to be adequately reconfigured to reduce or eliminate the gap. In some embodiments the inlay thickness at the edge is between about 2 microns and about 25 microns. In some embodiments the inlay thickness at the edge is between about 5 microns and about 20 microns. In some embodiments the inlay thickness at the edge is between about 7 microns and about 15 microns.

While the bevel surface angle is shown to be 30 degrees in the embodiment in FIG. 5C, the disclosure is not so limited. While making the angle 90 degrees would essentially create the peripheral region shown in the inlay in FIG. 4, the bevel surface can be at angles other than 30 degrees. In some embodiments the angle is between about 5 and about 70 degrees. In some embodiments the angle is between about 10 and about 50 degrees. In some embodiments it is between about 20 and 40 degrees.

As shown in FIG. 5C, there is a relatively small surface 60, which is optionally rounded, between anterior surface 52 and bevel surface 58. In this embodiment the surface has a radius of curvature of 2 microns, but in some embodiments is between about 2 microns and about 5 microns. After the peripheral region is reconfigured, the resultant peripheral edge thickness depends on the radius of rounded surface 60. In the example provided, the edge thickness changes from about 12 microns when the peripheral region is in the configuration shown in FIG. 5B to about 2-3 microns when the peripheral region is reconfigured as shown in FIG. 5C.

The configuration of the peripheral portion shown in FIG. 5C allows the inlay to be handled without damaging. If the edge were created with a sharp edge, it would be easy to damage the inlay while handling and it would be difficult to identify the correct orientation of inlay.

The inlay in FIGS. 5A-5C can be used to treat presbyopia, as is described in more detail in Application Nos. U.S. Pub. No. 2011/0218623, published on Sep. 8, 2011; U.S. Pat. No. 8,057,541, issued Nov. 15, 2011; U.S. Pub. No. 2008/0262610, published Oct. 23, 2008; and U.S. Pub. No. 2009/0198325, published Aug. 6, 2009.

In some embodiments the corneal implants are made of a hydrogel material.

Exemplary hydrogel materials from which the corneal implants described herein can be made are described in U.S. Pub. No. 2008/0262610, published Oct. 23, 2008.

In an alternative embodiment, the inlay from FIG. 3A-3B is modified to include beveled surface 58 shown in FIG. 3C. In this design the anterior surface includes a beveled surface as well. The bevel surface adjacent to the posterior surface, however, functions to eliminate the gap when the peripheral region is reconfigured.

The peripheral edge configuration shown in FIG. 5C can be incorporated into any suitable type of corneal inlay, regardless of the vision correction to be achieved. For example, the peripheral configuration can be incorporated into a multifocal inlay design. In some alternative embodiments a ring inlay to treat presbyopia can be modified to have a peripheral configuration shown in FIG. 5C. Both the inner and outer portions of a ring inlay can be modified to have the configuration shown in FIG. 5C.

The inlays described herein can be adapted to treat any visual error. For example, the inlays can be adapted to treat presbyopia in emmetropic, hyperopic, as well as myopic patients. Alternatively, the inlays can be used to treat corneal refractive errors such as hyperopia, or they can be modified to treat myopia or can be implanted with a pseudophakic monofocal intraocular lens. For example, the inlays described in U.S. Pat. No. 5,123,921 can be modified to include a beveled surface described herein.

1. A method of reducing gap formation between corneal tissue after the implantation of a corneal implant, comprising:
creating a corneal bed;
positioning a corneal implant on the corneal bed; and
reconfiguring a peripheral portion of the corneal implant from a first configuration to a second configuration.

2. The method of claim 1 wherein reconfiguring the peripheral portion comprises reconfiguring the peripheral portion so that it more closely conforms to the corneal bed.

3. The method of claim 2 wherein the reconfiguring step comprises reconfiguring the peripheral portion so that a first surface in the peripheral portion conforms more closely to the corneal bed when the peripheral portion is in the second configuration.

4. The method of claim 1 further comprising creating a corneal flap, lifting the corneal flap to create the corneal bed, and placing the flap back down over the corneal implant after positioning the corneal implant on the corneal bed,
wherein placing the flap back down over the corneal implant reconfigures the peripheral portion of the corneal implant from the first configuration to the second configuration such that gap formation between the flap, the corneal bed, and the cornea implant is reduced upon the reconfiguration to the second configuration.

5. The method of claim 1 wherein reconfiguring a peripheral portion comprises changing the orientation of a first surface from a first orientation to a second orientation so that it more closely conforms to the corneal bed.

6. The method of claim 5 wherein in the first orientation that first surface is at an angle relative to a posterior surface of the corneal implant.

7. The method of claim 5 wherein in the first configuration the surface is disposed at an angle less than 90 degrees relative to an axis that is orthogonal to an optical axis of the corneal implant.

8. The method of claim 7 wherein in the first configuration the surface is disposed at an angle between about 10 degrees and about 80 degrees relative to an axis that is orthogonal to an optical axis of the corneal implant.

9. The method of claim 1 wherein reconfiguring the peripheral portion comprises changing the orientation of a posterior beveled surface relative to a posterior surface of the corneal implant so that the beveled surface and is more closely aligned with the posterior surface.

10. The method of claim 1 wherein in the first configuration a peripheral edge of an anterior surface of the corneal implant is further from the corneal bed than the peripheral edge of the anterior surface in the second configuration.

11. A corneal implant with a peripheral portion adapted to reduce gap formation between corneal tissue after implantation of the corneal implant, the corneal implant comprising:
a body comprising a posterior surface adapted to be placed on a corneal bed, an anterior surface, and a peripheral portion comprising a first surface adapted to be reconfigured from a first orientation relative to the posterior surface to a second orientation relative to the posterior surface, wherein in the second orientation the first surface is more closely aligned with the posterior surface than in the first orientation.

12. The corneal implant of claim 11 wherein the first surface extends from the posterior surface and is disposed at an angle relative to the posterior surface in the first orientation.

13. The corneal implant of claim 12 wherein in the second orientation the first surface is disposed at a second angle relative to the posterior surface, wherein the second angle is less than the first angle.

14. The corneal implant of claim 13 wherein the second angle is substantially zero.

15. The corneal implant of claim 11 wherein in the first orientation the first surface is disposed at an angle less than 90 degrees relative to an axis orthogonal to an optical axis of the inlay.

16. The corneal implant of claim 15 wherein in the first orientation the first surface is disposed at an angle between about 10 degrees and about 80 degrees relative to an axis that is orthogonal to an optical axis of the corneal implant.

17. The corneal implant of claim 15 wherein in the first orientation the first surface is disposed at an angle between about 15 degrees and about 60 degrees relative to an axis that is orthogonal to an optical axis of the corneal implant.

18. The corneal implant of claim 16 wherein in the first orientation the first surface is disposed at an angle between less than 45 degrees relative to an axis that is orthogonal to an optical axis of the corneal implant.

19. The corneal implant of claim 11 wherein the corneal implant is made of a hydrogel material.

20. The corneal implant of claim 11 wherein the peripheral portion comprises an edge thickness of between about 10 and about 20 microns.

21. The corneal implant of claim 11 wherein the peripheral portion comprises an edge thickness that is less than about 30 microns.

22. The corneal inlay of claim 11 wherein the peripheral portion is adapted to be reconfigured when the corneal implant is implanted within corneal tissue less than or equal to about 50% of the depth of the corneal stroma.

23. A corneal implant adapted to reduce gap formation between corneal tissue after implanting the corneal implant, comprising
a body comprising a posterior surface adapted to be positioned on a corneal bed, a first surface disposed relative to the posterior surface at a first angle less than 90 degrees and greater than 0 degrees relative to an axis that is orthogonal to an optical axis of the body, and an anterior surface,
wherein the first surface is adapted to be reconfigured when implanted within corneal tissue such that it is at a second angle relative to the axis, wherein the second angle is less than the first angle.

24. A corneal implant adapted to reduce gap formation between corneal tissue after implanting the corneal implant, comprising
a body with a posterior surface adapted to be positioned on a corneal bed, an anterior surface, and a posterior portion that is adapted to be reconfigured from a first configuration to a second configuration upon implantation within corneal tissue,
wherein the peripheral portion comprises a first surface that is configured to more closely conform to the corneal bed when the peripheral portion is in the second configuration than when the peripheral portion in the first configuration.
